(19) United States
(12) Patent Application Publication
(10) Pub. No.: US 2016/0024489 A1
(43) Pub. Date: Jan. 28, 2016

(54) METHODS OF ISOLATING BACTERIAL STRAINS

(71) Applicant: THE BROAD OF REGENTS OF THE UNIVERSITY OF TEXAS SYSTEM, Austin, TX (US)

(72) Inventor: Philip SERWER, San Antonio, TX (US)

(73) Assignee: THE BROAD OF REGENTS OF THE UNIVERSITY OF TEXAS SYSTEM, Austin, TX (US)

(21) Appl. No.: 14/776,028

(22) PCT Filed: Mar. 14, 2014
(86) PCT No.: PCT/US14/27001

§ 371 Date: Sep. 14, 2015

Related U.S. Application Data
(60) Provisional application No. 61/785,392, filed on Mar. 14, 2013.

(51) Int. Cl. C12N 15/01; C12P 7/04

(52) U.S. Cl. CPC C12N 15/01; C12P 7/04

(57) ABSTRACT

Certain embodiments are directed to methods of developing bacterial strains having a selected metabolism for producing a target molecule(s) or bacterial strains or bacteriophage strains comprising a modified gene encoding a selected agent.

This application claims priority to U.S. Provisional Application Ser. No. 61/785,392 filed Mar. 14, 2013, which is incorporated herein by reference in its entirety.

### STATEMENT REGARDING FEDERALLY FUNDED RESEARCH

This invention was made with government support under GM24365 awarded by the National Institutes of Health. The government has certain rights in the invention.

### BACKGROUND

1. Field of the Invention

Embodiments of this invention are directed generally to biology and microbiology. Certain aspects are directed to co-evolution and production of microbes with a selected metabolism.

2. Background

The use of microbes in producing a number of useful molecules on a commercial scale has been a long-sought-after goal. Bioconversion of various molecules to particular products can use a variety of bacteria that have evolved the capacity to metabolize a substrate(s) to a target product. For example, methanotrophs can metabolize methane to methanol. Some bacterial genes can be evolved to have useful properties (conferring of resistance to herbicides, for example) that can be transferred to other organisms (food plants, for example). There remains a need to develop bacteria having improved characteristics for scaling and commercial production of such bacterial products.

### SUMMARY

Certain embodiments are directed to methods of accelerating development of bacterial strains having a selected metabolism for producing a target molecule(s), or bacterial strains or bacteriophage strains comprising a modified gene encoding a selected agent.

Certain embodiments are directed to accelerating the development of bacteria that produce a target molecule, e.g., produce methanol in commercial amounts. Target molecule-producing bacteria are bacteria that produce a target molecule in quantities that can be used in down stream processes. Down stream process can include, but are not limited to isolation of the target molecule or use of a medium containing the target molecule in a down stream process. Quantities of target molecules produced in the context of the described methods are increased over the amount produced by a non-modified or non-selected bacterial strain. In certain aspects the target molecule is produced at a level that is 10, 100, 1000, 10,000, or more times the starting bacterium. In certain aspects the amount is a commercially significant amount in that the cost of producing the target molecule is less that the value of the amount of target molecule produced. In certain aspects an inefficient target molecule-producing bacterial strain is exposed to co-evolution mixture under selective pressures. An inefficient target molecule-producing bacterial strain can be a strain of cells that produce low levels of the target molecule or harbor a metabolic pathway that can be altered to produce the target molecule when exposed to the appropriate selective pressures or methodology. Co-evolution or co-evolving refers to a process where the change or evolution of one biological entity is associated with and depends on a change in a second biological entity. Each biological entity in a co-evolutionary relationship exerts selective pressures on the other, thereby affecting each other's evolution. In certain aspects co-evolution occurs when certain viruses and their hosts are exposed to selective pressure. Viruses typically replicate more rapidly than their hosts and, therefore, evolve more rapidly. If virus-host gene exchange occurs, then the virus can accelerate the evolution of the host.

Applicant notes that selection can be used to produce essentially any molecule capable of being produced by a bacterium. In certain aspects the target molecule is methanol. In certain aspects the methods are initiated by selecting a methanol-dependent strain. A methanol dependent strain is a bacterium that can use only methanol as a source of carbon. Methanol dependent strains have been isolated and are known to exist, e.g., Methanomonas methylovora (Kouno et al., J. Gen. Appl. Microbiol. 19:11-21, 1973).

Certain aspects use at least two co-dependent bacterial strains. The term “co-dependent” refers to bacterial strains that rely on a metabolite or nutrient produced by the other bacterial strain for growth and reproduction. The term “rely”, as used in the context of co-dependency, means that in the absence of such metabolite or nutrient the dependent bacterial strains grows or replicates at a significantly lower level or not at all. In certain aspects at 3, 4, 5 or more bacteria can be used in the co-evolution compositions. In a further aspect additional bacteriophage can be included in the co-evolution compositions. The additional bacteriophage can be specific for other bacterium in the mixture, such as the metabolite-dependent bacterium. In other aspects more than on one bacteriophage can be present for a single bacterium. In still a further aspect more than one bacterial strain can be susceptible to a single bacteriophage.

Certain embodiments are directed to co-evolving fermentation compositions comprising (a) a metabolite-dependent bacterium whose propagation is dependent on a first metabolite, (b) a metabolite-producing bacterium that produces the first metabolite and requires an essential nutrient for propagation, and (c) a co-evolving bacteriophage that propagates in and co-evolves with the metabolite-producing bacterium. In certain aspects a metabolite can be methanol or another carbon source. In a further aspect the metabolite-producing bacteria is an auxotroph, e.g., an amino acid or fatty acid auxotroph. In certain aspects the bacteriophage is a member of the Myoviridae. In a further aspect the bacteriophage is a long-genome bacteriophage. A long-genome bacteriophage has a genome of at least 150, 200, 250, 300 or more kilobases (Kb). In certain aspects the bacteriophage can be engineered to encode 1, 2, 3, 4, 5, or more proteins or enzymes (endogenous or heterologous proteins or enzymes) that can be utilized or evolved to produce a metabolite.

Certain embodiments are directed to methods of developing a bacterial strain that produces a target metabolite, comprising the steps of: (a) Exposing at least two co-dependent bacterial strains and a co-evolving bacteriophage to a selection medium having an initial concentration of exogenous target metabolite, wherein a first bacterium is a target metabolite-dependent bacterium and a second bacterium is a target metabolite-producing bacterium. The co-evolving bacteriophage is selective for the target metabolite-producing bacterium; (b) incubating the selection medium comprising the co-dependent bacteria and co-evolving bacteriophage; (c) reducing the concentration of exogenous target metabolite; and (d) isolating a second target metabolite-producing bacterium (evolved bacterium) that has evolved from the first target metabolite-producing bacterium, the evolved bacterium produces the target metabolite at levels greater than the first target metabolite-producing bacterium. The methods can further comprise exposing the selection medium containing the bacteria and bacteriophage to a mutagenic agent. The mutagenic agent can be a chemical mutagen or electromagnetic radiation. In certain aspects the target metabolite is methanol.

Other embodiments are directed to methods of producing methanol comprising (a) incubating or fermenting a production medium containing an evolved methanol-producing bacteria; and (b) isolating methanol from the production medium.

Certain embodiments are directed to producing a variant of a target gene, which can encode an enzyme that modifies or metabolizes a selective agent, e.g., herbicide resistance gene and its encoded protein. Gene selection can comprise the steps of (a) exposing one or more bacterial strains and one or more co-evolving bacteriophages to a selection medium having an initial concentration of a selective agent, e.g., herbicide. In the case of a single bacterium and its co-evolving bacteriophage, incubation is in a selection medium comprising an initial amount of a selective agent that may be increased as a resistance gene evolves in both the co-evolving bacteriophage and its host. The high evolution speed of the bacteriophage increases the evolution speed for the host via gene transfer. In certain aspects a single gene encodes a protein that modifies the selective agent and renders it less toxic to the bacteria. Over time the concentration of the selective agent is increased. After a predetermined amount of time or reaching a predetermined concentration of selective agent the co-evolved target gene is isolated and characterized. This gene can now be transferred to other organisms to produce resistance to the toxic agent. The methods can further comprise exposing the selection medium to a mutagenic agent. The mutagenic agent can be a chemical mutagen or electromagnetic radiation.

Other embodiments of the invention are discussed throughout this application. Any embodiment discussed with respect to one aspect of the invention applies to other aspects of the invention as well and vice versa. The embodiments in the Example section are understood to be embodiments of the invention that are applicable to all aspects of the invention.

The terms “inhibiting,” “reducing,” or “prevention,” or any variation of these terms, when used in the claims and/or the specification, includes any measurable decrease or complete inhibition to achieve a desired result.

The terms “stimulate,” “increase,” or “enhance,” or any variation of these terms, when used in the claims and/or the specification, includes any measurable increase or acquisition of ability to achieve a desired result.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed herein can be implemented with respect to any method or composition of the invention, and vice versa. Furthermore, compositions and kits of the invention can be used to achieve methods of the invention.

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.” It is also contemplated that anything listed using the term “or” may also be specifically excluded.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION

Embodiments of the invention are directed to methods and compositions for development of bacteria that produce or have an enhanced production of a target molecule. An example of such methods is provided for the production of methanol from methane. Applicant provides this particular application as demonstrating the methodology. The methods described herein are not intended to be limited to methanol production, but can be further modified to produce any number of other molecules.

In certain aspects the target molecule is methanol. Applicants note that essentially any molecule capable of being produced by a bacterium can be selected. In certain aspects the methods are initiated by selecting a methanol-dependent strain. A methanol dependent strain is a bacterium that can use only methanol as a source of carbon. Methanol dependent strains have been isolated and are known to exist, e.g., Methanomonas methylovora (Kouno et al., J. Gen. Appl. Microbiol. 19:11-21, 1973).

In certain [other] aspects a methanol-producing bacterial strain is selected for targeted evolution. The methanol-producing strain (typically a methanotroph) synthesizes methanol, but at levels that are not high enough for commercial production. In certain aspects the strain must also not produce essential metabolite(s) provided by the methanol-dependent strain. One example of such a methanol producing bacterium is Methylococcus capsulatus.

In certain aspects a bacteriophage specific for the methanol-producing bacterial strain is introduced to accelerate evolution of the methanol-producing bacteria. Bacteriophages for Methylosinus trichosporium have previously been isolated (Tiutikov et al., Mikrobiologiia 45:1056-1062, 1976). One option to isolate such bacteriophages, particularly large bacteriophages, is to elute such bacteriophage from biofilms. Possible biofilms include those associated with mud and/or muck in livestock pens and water troughs, as well analogous natural environments.

The evolution of the methanol-producing bacteria can be accelerated by using co-evolution with a compatible bacteriophage. Co-evolution can be evaluated or assessed by using DNA analysis of the bacteria, the bacteriophage, or both the bacteria and the bacteriophage. DNA analysis can comprise a variety of DNA analysis techniques including but not limited to Southern blotting, nucleic acid amplification, nucleic acid arrays, and nucleic acid sequencing. In certain embodiments the bacterial genome and/or the bacteriophage genome can be fully or partially sequenced using any of a number of sequencing technologies. In certain aspects pyrophosphate-based sequencing (pyrosequencing) can be used. Thus induced sequence changes can be documented by DNA analysis.

In a further aspect, the methanol-producing strain can be altered rendering it dependent on an essential compound (i.e., engineering auxotrophy) or alternatively selecting an established auxotroph. In certain aspects the methanol-dependent strain can either produce or be altered to produce the essential compound required by the methanol-producing strain. In certain embodiments the essential compound can be an amino acid, vitamin, fatty acid, or the like.

In one embodiment a first bacterial mixture is produced that includes a methanol-producing strain, co-evolving bacteriophage specific for the methanol-producing strain, and an essential nutrient for the methanol-producing strain. A methanol-dependent strain and an initial amount of methanol is then added to the first bacterial mixture. Applicants note that each of these components may be added at different times and in a different order as long as a functional selective bacterial mixture is the end product. The concentrations of both methanol and essential nutrient are progressively decreased, thereby progressively selecting for a methanol-producing strain that hyper-produces methanol because methanol is needed by the methanol-dependent bacterial strain in order to produce the depleted nutrient needed by the methanol-producing strain. That is to say, the two bacteria will co-evolve with co-evolution rate increased by the bacteriophage. Tests for methanol production can be performed throughout the three-microbe-dependent co-evolution process.

### I. Bacterial Strains

Bacteria constitute a large domain of prokaryotic microorganisms. Typically a few micrometers in length, bacteria have a wide range of shapes, ranging from spheres to rods and spirals. Bacteria are present in most habitats on Earth growing, for example, in soil, water, acidic hot springs, radioactive waste, and deep in the Earth's crust, as well as in organic matter and the live bodies of plants and animals. Bacteria are vital in recycling nutrients, with many steps in nutrient cycles depending on these organisms, such as the fixation of nitrogen from the atmosphere and putrefaction.

Certain embodiments of the invention utilize co-dependent bacteria to increase the level of a chosen metabolite produced by a bacterium. In certain aspects co-dependent bacteria include (a) a first (metabolite-dependent) bacterial strain that is dependent on a metabolite produced from a second strain that produces the metabolite (metabolite-producing bacterial strain), and (b) a second (metabolite-producing) bacterial strain that is an auxotroph that requires an essential nutrient produced by the first, metabolite-dependent strain. In certain aspects the metabolite is a targeted molecule for which the second bacteria are to be modified to produce the molecule at increased levels. The essential nutrient will be a compound needed to sustain the metabolite-producing bacteria, but it is not necessary to modify the production levels of the essential nutrient by the first metabolite dependent bacteria.

Bacteria can be classified on the basis of cell structure, cellular metabolism or on differences in cell components such as DNA, fatty acids, pigments, antigens, and quinones. While these schemes allowed the identification and classification of bacterial strains, it was unclear whether these differences represented variation between distinct species or between strains of the same species. This uncertainty was due to the lack of distinctive structures in most bacteria, as well as lateral gene transfer between unrelated species. Due to lateral gene transfer, some closely related bacteria can have very different morphologies and metabolisms. Modern bacterial classification emphasizes molecular systematics, using molecular biology-based techniques such as guanine/adenine ratio determination, genome-genome hybridization, as well as nucleic acid sequencing. Classification of bacteria is determined by publication in the International Journal of Systematic Bacteriology and Bergey's Manual of Systematic Bacteriology. The International Committee on Systematic Bacteriology (ICSB) maintains international rules for the naming of bacteria and taxonomic categories and for the ranking of them in the International Code of Nomenclature of Bacteria.

The Gram stain, developed in 1884 by Hans Christian Gram, characterizes bacteria based on the structural characteristics of their cell walls. The thick layers of peptidoglycan in the “Gram-positive” cell wall stain purple, while the thin “Gram-negative” cell wall appears pink. By combining morphology and Gram-staining, most bacteria can be classified as belonging to one of four groups (Gram-positive cocci, Gram-positive bacilli, Gram-negative cocci and Gram-negative bacilli). Some organisms are best identified by stains other than the Gram stain, particularly mycobacteria or Nocardia, which show acid-fastness on Ziehl-Neelsen or similar stains. Other organisms may need to be identified by their growth in special media, or by other techniques, such as serology.

Bacteria for use in the invention can be almost any bacteria having the basic characteristic to evolve a targeted phenotype. Bacteria used in the invention can include, but are not limited to bacteria selected from Family Acidobacteriaceae; Family Acidimicrobiaceae; Family Actinomycetaceae; Family Actinomycetaceae; Family Corynebacteriaceae; Family Gordoniaceae; Family Mycobacteriaceae; Family Nocardiaceae; Family Tsukamurellaceae; Family Williamsiaceae; Family Acidothermaceae; Family Frankiaceae; Family Geodermatophilaceae; Family Kineosporiaceae; Family Microsphaeraceae; Family Sporichthyaceae; Family Glycomycetaceae; Family Beutenbergiaceae; Family Bogoriellaceae; Family Brevibacteriaceae; Family Cellulomonadaceae; Family Dermabacteraceae; Family Dermatophilaceae; Family Dermacoccaceae; Family Intrasporangiaceae; Family Jonesiaceae; Family Microbacteriaceae; Family Micrococcaceae; Family Promicromonosporaceae; Family Rarobacteraceae; Family Sanguibacteraceae; Family Micromonosporaceae; Family Nocardioidaceae; Family Propionibacteriaceae; Family Actinosynnemataceae; Family Pseudonocardiaceae; Family Streptomycetaceae; Family Nocardiopsaceae; Family Streptosporangiaceae; Family Thermomonosporaceae; Family Bifidobacteriaceae; Family Coriobacteriaceae; Family Rubrobacteraceae; Family Sphaerobacteraceae; Family Aquificaceae; Family Hydrogenothermaceae; Family Bacteroidaceae; Family Rikenellaceae; Family Prevotellaceae; Family Flavobacteriaceae; Family Myroidaceae; Family Blattabacteriaceae; Family Sphingobacteriaceae; Family Saprospiraceae; Family Flexibacteraceae; Family Flammeovirgaceae; Family Crenotrichaceae; Family Chlamydiaceae; Family Parachlamydiaceae; Family Rhabdochlamydiaceae; Family Simkaniaceae; Family Waddliaceae; Family Chlorobiaceae; Family Chrysiogenaceae; Family Deferribacteraceae; Family Dictyoglomaceae; Family Alicyclobacillaceae; Family Bacillaceae; Family Caryophanaceae; Family Listeriaceae; Family Paenibacillaceae; Family Planococcaceae; Family Sporolactobacillaceae; Family Staphylococcaceae; Family Thermoactinomycetaceae; Family Turicibacteraceae; Family Acidaminococcaceae; Family Clostridiaceae; Family Eubacteriaceae; Family Heliobacteriaceae; Family Lachnospiraceae; Family Peptococcaceae; Family Peptostreptococcaceae; Family Syntrophomonadaceae; Family Halanaerobiaceae; Family Halobacteroidaceae; Family Thermoanaerobacteriaceae; Family Thermodesulfobiaceae; Family Mycoplasmataceae; Family Entomoplasmataceae; Family Spiroplasmataceae; Family Anaeroplasmataceae; Family Erysipelotrichaceae; Family Acholeplasmataceae; Family Fusobacteriaceae; Family Planctomycetacea; Family Caulobacteraceae; Family Kordiimonadaceae; Family Parvularculaceae; Family Aurantimonadaceae; Family Bartonellaceae; Family Beijerinckiaceae; Family Bradyrhizobiaceae; Family Brucellaceae; Family Hyphomicrobiaceae; Family Methylobacteriaceae; Family Phyllobacteriaceae; Family Rhizobiaceae; Family Rhodobiaceae; Family Rhodobacteraceae; Family Rhodospirillaceae; Family Rhodospirillaceae; Family Rickettsiaceae; Family Ehrlichiaceae; Family Holosporaceae; Family Sphingomonadaceae; Family Alcaligenaceae; Family Burkholderiaceae; Family Comamonadaceae; Family Oxalobacteraceae; Family Hydrogenophilales; Family Methylophilaceae; Family Spirochetaceae; Family Serpulinaceae; Family Leptospiraceae; Family Thermodesulfobacteriaceae; Family Thermotogaceae; or Family Verrucomicrobiaceae.

A. Metabolite Dependent Bacteria

In the context of the described methods, a metabolite dependent bacterium is a bacterium that is dependent upon a particular molecule(s) that may be (but is not necessarily) produced by the bacteria targeted for evolution, e.g., the metabolite-producing bacteria. In certain aspects the metabolite-dependent bacterium is a methanol-dependent bacterium.

B. Metabolite Producing Bacteria

In the context of the described methods, a metabolite producing bacterium is a bacterium that has some capacity to produce a target molecule, which may or may not be the metabolite on which the metabolite dependent bacteria requires. In certain aspects the metabolite-producing bacterium may be an auxotroph. In certain aspects an auxotroph can require alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, valine/isoleucine/leucine in order to propagate.

### II. Bacteriophage

A bacteriophage (phage) is a virus that infects and replicates within bacteria. Bacteriophages are composed of proteins that encapsulate a DNA or RNA genome, and may have relatively simple or elaborate structures. Their genomes may encode as few as four genes, and as many as hundreds of genes. Bacteriophage replicate within bacteria following the injection of their genome into the bacterial cytoplasm.

Bacteriophages are widely distributed in locations populated by bacterial hosts, locations that include soil and the intestines of animals. Even sea water, has up to 9×10⁸ virions per milliliter in microbial mats at the surface (Wommack and Colwell, Microbiology and Molecular Biology Reviews 64 (1): 69-114, 2000). Biofilms can have at least one million times more bacteriophage per volume than sea water, based on electron microscopy of thin sections. Thus, in certain aspects biofilms can be used as a source for bacteriophage. Bacteriophage have been used as an alternative to antibiotics and are seen as a possible therapy against multi-drug-resistant strains of bacteria.

The dsDNA tailed bacteriophages, or Caudovirales, account for 95% of bacteriophages reported in the scientific literature. Other bacteriophages occur in the biosphere, with different protein components (capsids), genomes, and lifestyles. Bacteriophages are classified according to morphology and nucleic acid by the International Committee on Taxonomy of Viruses (ICTV). Currently there are at least nineteen families of bacteriophage recognized. Of these, only two families have RNA genomes and only five families are enveloped by a membrane. Of the viral families with DNA genomes, only two have single-stranded genomes. Eight of the viral families with DNA genomes have circular genomes, while nine have linear genomes. Nine families infect bacteria only, nine infect archaea only, and one (Tectiviridae) infects both bacteria and archaea. Families of bacteriophage include Myoviridae, Siphoviridae, Podoviridae, Lipothrixviridae, Rudiviridae, Ampullaviridae, Bicaudaviridae, Clavaviridae, Corticoviridae, Cystoviridae, Fuselloviridae, Globuloviridae, Guttavirus, Inoviridae, Leviviridae, Microviridae, Plasmaviridae, and Tectiviridae. One or more bacteriophage can be selected for use in the methods described herein.

Current data indicate that roughly 10³¹ bacteriophages exist worldwide, including about 10⁸ genotypes and possibly most of the earth's gene diversity as estimated by metagenomics and fluorescence and electron microscopy (Breitbart and Rohwer Trends Microbiol 13:278-84, 2005; Brüssow and Kutter Phage ecology. In Bacteriophages: Biology and Applications Edited by: Kutter and Sulakvelidze, Boca Raton, Fla.: CRC Press; pages 129-63, 2005; Rohwer, Cell 113:141, 2003; Williamson et al., Appl Environ Microbiol 71:3119-25, 2005). Less than 1% of the observed bacteriophages have ever been grown in culture (sometimes called “the great plaque count anomaly”). The great plaque count anomaly is especially dramatic in the case of soil-borne bacteriophages. Propagated bacteriophages are sometimes not obtained from soil samples in spite of bacteriophage concentrations in the 10⁸-10⁹ range per gram, when detected by microscopy (Ashelford et al., Appl Environ Microbiol 69:285-89, 2003). Some bacteriophages, though viable, are probably not detected by any past procedures. Genomes of currently unpropagated bacteriophages are potentially a major source of unexplored environmental gene diversity.

Knowledge of environmental virus gene diversity has been recently expanded by sequencing of large eukaryotic phycodnaviruses and related viruses. These viruses have double-stranded DNA genomes with a length between 200 and 1,200 Kb (Claverie et al., Virus Res 117:133-44, 2006; Dunigan et al., Virus Res 117:119-32, 2006; Ghedin and Fraser, Trends Microbiol 13:56-57, 2005; Iyer et al., Virus Res 117:156-84, 2006). Large double-stranded DNA bacteriophages also exist, including Bacillus megaterium bacteriophage G (˜670 Kb genome (Hutson et al., Biopolymers 35:297-306, 1995)), Pseudomonas aeruginosa bacteriophage φKZ (280 Kb genome (Mesyanzhinov et al., J Mol Biol 317:1-19, 2002)) and several bacteriophages that are relatives of bacteriophage T4 by the criteria of DNA replication/recombination strategy, structure and interface of DNA replication to DNA packaging (Petrov et al., J Mol Biol 361:46-68, 2006; Nolan et al., Virol J 3:30, 2006).

However, of the 5,400 or so bacteriophages that have been isolated (Ackermann, Classification of bacteriophages. In The bacteriophages Edited by: Calendar R. Oxford: Oxford University Press 8-16, 2006), 96% have double-stranded DNA genomes and of 405 deposited in databases, only 6 have genomes as long as 200 Kb. Two other T4-like bacteriophage genomes in draft status are also in this range (Petrov et al., J Mol Biol 361:46-68, 2006). Statistical analysis reveals a significant undersampling of long-genome bacteriophages (Claverie et al., Virus Res 117:133-44, 2006). The strong possibility exists that long-genome bacteriophages (>200 Kb genome) are more frequent and are major contributors to microbial ecology, but are under-sampled because of the use of classical bacteriophage propagation procedures and possibly also classical processing of environmental samples for microscopy. For example, bacteriophage G was discovered by accident ˜40 years ago through electron microscopy of a preparation of another bacteriophage (Donelli, Atti Accad Naz Lincei-Rend Clas Sci Fis Mat Nat 44:95-97, 1968). Long-genome bacteriophages are of interest for use in host/bacteriophage co-evolution.

To identify long-genome environmental bacteriophages, extraction and propagation can be performed in comparatively dilute agarose gels (e.g., 0.15% agarose gels). In certain aspects gels can contain nutrients or nutrient medium, such as 10 g Bacto tryptone, 5 g KCl in 1000 ml water with 0.002 M CaCl₂ (Serwer et al., Virology 329:412-24, 2004). Bacteriophages can be screened using single plaque cloning and determining the change in plaque size with change in supporting agarose gel concentration. For example, Bacillus thuringiensis bacteriophage 0305φ8-36 made small (<1 mm) plaques in a 0.4% agarose supporting gel. Plaques became progressively larger as the agarose gel concentration decreased to 0.2% and 0.15%. This dependence is comparatively steep, as confirmed in a side-by-side comparison with bacteriophages T4 and G. Post-isolation, 0305φ8-36 grew only in gels of either 0.25% or more dilute agarose (Serwer et al., Virol J (2007), 4:21).

Further analysis of bacteriophage 0305φ8-36 confirmed that it was a large bacteriophage. Electron microscopy of a negatively stained specimen of purified bacteriophage particles revealed a contractile-tail virus (myovirus) (Bradley, Bacteriol Rev 31:230-314, 1967; Fauquet et al. (Eds): Virus Taxonomy: The Eighth Report of the International Committee on Taxonomy of Viruses San Diego: Academic Press; 2005) with a polyhedral DNA-containing capsid that had a diameter of 95±4 nm. In addition, bacteriophage 0305φ8-36 has (a) a long tail of 486±23 nm in length and 26±3 nm in diameter, and (b) tail fibers that were also comparatively large, 187±13 nm in length and 10±1 nm in diameter. Bacteriophage tail fiber diameter has been generally conserved at about 2 nm among other tailed bacteriophages (Ackermann, Adv Virus Res 51:135-201, 2000). In addition, the tail fibers had an unusual sine wave-like appearance in projection and are presumably corkscrew-like in three dimensions. The genome of 0305φ8-36 was approximately 221 Kb as determined by pulsed field gel electrophoresis (PFGE). Bacteriophages with morphology of this general type have been reported (Ackermann et al., Can J Microbiol 41:294-297, 1995).

The unusual biology of 0305φ8-36 is accompanied by an unusual genome, based on sequence determination. For example, the 0305φ8-36 DNA packaging ATPase was identified by use of the SAM HMM procedures previously described (Serwer et al., Virology 329:412-24, 2004) with E=5.17e-54. Motifs found and aligned include: (1) ATPase motif, including adenine-binding motif, P-loop motif, and DExx box and (2) conserved aspartate residues of the endonuclease ruvC fold. The aligned 0305φ8-36 DNA packaging ATPase intersects the homology tree for this protein only at the center. That is to say, no other known DNA packaging ATPase is in the same class. Most other genes are too diverged from known genes to identify. A few 0305φ8-36 genes for myovirus structural components have been identified, but without any indication of membership in any previously known group.

Large bacteriophages like 0305φ8-36 are best isolated and propagated using dilute gel propagation because the classical detection procedures, i.e., community sequencing (Rodriguez-Brito et al., BMC Bioinformatics 7:162, 2006), liquid enrichment culture, and microscopy (Carlson, Appendix: Working with bacteriophages: Common techniques and methodological approaches. In Bacteriophages: Biology and Applications, Edited by: Kutter and Sulakvelidze Boca Raton, Fla.: CRC Press 437-494, 2005), are not expected to work with many of them.

Laboratory bacteriophage/host co-evolution does not occur with most bacteriophages, but does with some. A natural co-evolution process has been previously described, for an example see Macia et al. Nature 450:1079-81, 2007. Laboratory bacteriophage/host co-evolution has not been used to develop a bacterial strain with a targeted metabolic pathway or to produce a targeted metabolite. Bacteriophage 0305 φ 8-36 undergoes one round of co-evolution with its host (Serwer et al. Virology Journal 4: 21, 2007) and has been found subsequently to undergo at least 10 more.

In certain aspects the bacteriophage is a large bacteriophage. In a further aspect, the bacteriophage is co-evolving with a metabolite-producing bacterial strain. In certain aspects the combination of metabolite producing bacteria and associated co-evolving bacteriophage can be isolated from environmental samples. The environmental samples can be obtained from the same or different location.

1. A co-evolving fermentation composition comprising (a) a metabolite-dependent bacterium whose propagation is dependent on a first metabolite, (b) a metabolite-producing bacterium that produces the first metabolite and requires an essential nutrient for propagation, and (c) a co-evolving bacteriophage that propagates in and co-evolves with the metabolite-producing bacterium.

2. The composition of claim 1, wherein the metabolite-dependent bacterium produces an essential nutrient for the metabolite-producing bacterium.

3. The composition of claim 1, wherein the metabolite is methanol.

4. The composition of claim 1, wherein the metabolite-producing bacterium is an auxotroph.

5. The composition of claim 4, wherein the auxotroph is an amino acid auxotroph.

6. The composition of claim 1, wherein the bacteriophage is a Myoviridae.

7. The composition of claim 6, wherein the bacteriophage is a long-genome bacteriophage.

8. The composition of claim 7, wherein the long-genome bacteriophage has a genome of at least 200 kilobases (Kb).

9. The composition of claim 1, further comprising a second bacteriophage that propagates in the metabolite-dependent bacterium.

10. A method of developing a bacterial strain that produces a target metabolite comprising:
(a) exposing at least two co-dependent bacterial strains and at least one co-evolving bacteriophage to a selection medium having an initial concentration of exogenous target metabolite, wherein a first bacterium is target metabolite-dependent bacterium and a second bacterium is a first target metabolite-producing bacterium, and the co-evolving bacteriophage is selective for the target metabolite-producing bacterium;
(b) incubating the selection medium comprising the co-dependent bacteria and co-evolving bacteriophage;
(c) reducing the concentration of exogenous target metabolite; and
(d) isolating a second target metabolite-producing bacterium that has evolved from the first target metabolite-producing bacterium that produces the target metabolite at levels greater than the first target metabolite-producing bacterium.

11. The method of claim 10, further comprising exposing the selection medium to a mutagenic agent.

12. The method of claim 11, wherein the mutagenic agent is a chemical mutagen or electromagnetic radiation.

13. The method of claim 10, wherein the target metabolite is methanol.

14. A method of producing methanol comprising:
(a) incubating a production medium containing an evolved methanol-producing bacteria of claim 13; and
(b) isolating methanol from the production medium.
